(19) United States
(12) Patent Application Publication
(10) Pub. No.: US 2024/0000705 A1
(43) Pub. Date: Jan. 4, 2024

(54) USE OF SURFACTANT WITH HIGH MOLECULAR WEIGHT FISH GELATIN BASED DOSAGE FORMULATIONS TO IMPROVE FLOW CHARACTERISTICS

(71) Applicant: Catalent U.K. Swindon Zydis Limited, Glasgow (GB)

(72) Inventor: Leon Paul GROTHER, Swindon, Wiltshire (GB)

(73) Assignee: Catalent U.K. Swindon Zydis Limited, Glasgow (GB)

(21) Appl. No.: 18/026,529

(22) PCT Filed: Sep. 17, 2021
(86) PCT No.: PCT/EP2021/075710

§ 371 Date: Mar. 15, 2023

Related U.S. Application Data
(60) Provisional application No. 63/079,852, filed on Sep. 17, 2020.

(51) Int. Cl. A61K 9/00; A61K 47/42; A61K 47/34; A61K 47/26; A61K 38/095; A61K 31/40; A61K 9/19; A61K 47/12

(52) U.S. Cl. CPC A61K 9/0056; A61K 47/42; A61K 47/34; A61K 47/26; A61K 38/095; A61K 31/40; A61K 9/19; A61K 47/12

(57) ABSTRACT

The present disclosure is directed to use of surfactant with fish gelatin based, freeze dried orally disintegrating tablets. Specifically, Applicants discovered that a small amount of surfactant in combination with high molecular weight fish gelatin in a pharmaceutical formulation can ensure good solution/suspension flow into preformed molds during dosing in order that the finished dosage form has an acceptable shape.

### CROSS-REFERENCE TO RELATED APPLICATIONS

This application is a national stage application under 35 U.S.C. § 371 of International Application No. PCT/EP2021/075710, filed Sep. 17, 2021, which claims priority to and the benefit of U.S. Provisional Application No. 63/079,852, filed Sep. 17, 2020, the entire contents of each priority application is incorporated herein by reference.

### FIELD OF THE DISCLOSURE

This disclosure relates to pharmaceutical compositions that can freely flow into a preformed mold during dosing. More specifically, this disclosure relates to pharmaceutical formulations comprising a small amount of a suitable surfactant that can reduce the surface tension of the formulation and allow it to freely flow into the preformed mold during dosing.

### BACKGROUND

The process of manufacturing dosage forms for the delivery of an active pharmaceutical ingredient (“API”) includes the step of dosing a pharmaceutical formulation into a preformed mold. As used herein, “dosed” or “dosing” refers to the deposition of a pre-determined aliquot of solution or suspension. As used herein, “preformed mold” refers to any suitable container or compartment into which an aqueous solution or suspension may be deposited and within which subsequently freeze dried.

The pharmaceutical formulations that are dosed into the preformed molds can include a matrix former that provides the network structure of the dosage form that imparts strength and resilience to the dosage form during handling. One such matrix former is high molecular weight fish gelatin. High molecular weight fish gelatin is defined as a fish gelatin in which more than 50% of the molecular weight distribution is greater than 30,000 Daltons.

### SUMMARY

Pharmaceutical formulations (i.e., liquid solutions or suspensions) with the gelatin (which itself is known to have surface active properties) are expected to flow well when dosed into a preformed mold due to their low viscosity (<50 mPa s). However, Applicants unexpectedly found that pharmaceutical formulations comprising high levels (e.g., 5 wt. % or greater) of high molecular weight fish gelatin have surprisingly poor flow properties that can result in poorly shaped dosage forms due to the formulation not providing full coverage of the preformed mold during dosing and prior to freeze drying. For example, FIG. 3 illustrates misshaped dosage forms containing high molecular weight fish gelatin. As such, 100% surface inspection can be required. A 100% surface inspection can include inspecting every unit (i.e., dosage form) visually. Conversely, under normal circumstances when misshapen dosage forms are an anomaly, only a small selection of units (i.e., dosage forms) can be visually inspected. Thus, a 100% surface inspection rate can add cost to the production process from man hours required for the inspection as well as dosage forms that are discarded due to poor shape.

Attempts to overcome the poor flow properties of the pharmaceutical formulations during dosing have been conducted using other gelatins with better flow properties than high molecular weight fish gelatin. However, these alternative gelatins are not always a preferred choice because bovine and porcine gelatins may not be suitable to vegetarian, vegan, or certain religious groups. In addition, fish gelatins with lower molecular weight (i.e., fish gelatin in which more than 50% of the molecular weight distribution is below 30,000 Daltons) can cause surface defects (e.g., nodules) on the dosage forms, thereby also requiring a 100% surface inspection and the costs that go along with it.

Another potential solution to the problem of poor flow properties of the pharmaceutical formulations during dosing is to formulate the dosage form using a larger volume. The increased volume and weight of the dosing formulation can force the formulation to fill the preformed mold as the increase in weight can overcome the surface tension that may otherwise prevent the formulation from flowing over the entire bottom surface of the preformed mold. However, increasing the volume and weight is an additional cost added due to raw material costs (e.g., ingredient costs, packaging costs) and processing costs (e.g., increased freeze drying time).

Accordingly, Applicants have discovered that the addition of a small amount of a suitable surfactant (e.g., poloxamer 188, sodium lauryl sulfate, docusate sodium) to the dosing pharmaceutical formulation can reduce the surface tension of the formulation and allow it to freely flow into the preformed mold and cover the bottom most surface or base of the preformed mold. This can result in a well-shaped dosage form.

In some embodiments, a pharmaceutical formulation for preparing a pharmaceutical dosage form includes an active pharmaceutical ingredient; 0.01-0.3 wt. % of a surfactant; 4-6 wt. % of high molecular weight fish gelatin; and a structure former. In some embodiments, the surfactant comprises 0.05-0.2 wt. % of the pharmaceutical formulation. In some embodiments, the surfactant is a non-ionic surfactant. In some embodiments, the non-ionic surfactant comprises polyoxyethylene-polyoxypropylene copolymer. In some embodiments, the surfactant is poloxamer 188. In some embodiments, the surfactant is an anionic surfactant. In some embodiments, the anionic surfactant comprises one or more of sodium lauryl sulfate and docusate sodium. In some embodiments, the pharmaceutical formulation comprises 4.5-5.5 wt. % of the high molecular weight fish gelatin. In some embodiments, the pharmaceutical formulation comprises 3-5 wt. % of the structure former. In some embodiments, the structure former comprises mannitol. In some embodiments, the formulation includes a pH modifier. In some embodiments, the pH modifier comprises citric acid, maleic acid, tartaric acid, or hydrochloric acid. In some embodiments, the pH of the pharmaceutical formulation is 4-6. In some embodiments, the solvent comprises water. In some embodiments, the active pharmaceutical ingredient comprises one or more of desmopressin and glycopyrrolate. In some embodiments, the formulation has a viscosity of 9-12 mPa s. In some embodiments, the formulation has a relative density of 1.2-1.3. In some embodiments, the formulation has a surface tension of 60-80 mN/m.

In some embodiments, a method of producing a freeze-dried dosage form for the delivery of an active pharmaceutical ingredient includes: dosing a pharmaceutical formulation into a preformed mold, wherein the pharmaceutical formulation comprises: an active pharmaceutical ingredient; 0.01-0.3 wt. % of a surfactant; 4-6 wt. % of high molecular weight fish gelatin; and a structure former; and freeze-drying the dosed pharmaceutical formulation to form the dosage form. In some embodiments, the method includes freezing the dosed pharmaceutical formulation at a temperature of −40° C. to −120° C. In some embodiments, the method includes annealing the frozen pharmaceutical formulation by holding it at a temperature of less than −25° C. for 0.25-3 hours. In some embodiments, the dosed pharmaceutical formulation is frozen at a temperature of −50° C. to −70° C. for a duration of about 1-5 minutes. In some embodiments, the surfactant comprises 0.05-0.2 wt. % of the pharmaceutical formulation. In some embodiments, the surfactant is a non-ionic surfactant. In some embodiments, the non-ionic surfactant comprises polyoxyethylene-polyoxypropylene copolymer. In some embodiments, the surfactant is poloxamer 188. In some embodiments, the pharmaceutical formulation comprises 4.5-5.5 wt. % of the high molecular weight fish gelatin. In some embodiments, the pharmaceutical formulation comprises 3-5 wt. % of the structure former. In some embodiments, the structure former comprises mannitol. In some embodiments, the pharmaceutical formulation comprises a pH modifier. In some embodiments, the pH modifier comprises citric acid, maleic acid, tartaric acid or hydrochloric acid. In some embodiments, the pH of the pharmaceutical formulation is 4-6. In some embodiments, the pharmaceutical formulation comprises a solvent. In some embodiments, the solvent comprises water. In some embodiments, the active pharmaceutical ingredient comprises one or more of desmopressin and glycopyrrolate. In some embodiments, a wet filling dosing weight of the pharmaceutical formulation is less than or equal to 200 mg. In some embodiments, the formulation has a viscosity of 9-12 mPa s. In some embodiments, the formulation has a relative density of 1.2-1.3. In some embodiments, the formulation has a surface tension of 60-80 mN/m.

In some embodiments, a dosage form for the delivery of an active pharmaceutical ingredient prepared by a process comprising: dosing a pharmaceutical formulation into a preformed mold, wherein the pharmaceutical formulation comprises: an active pharmaceutical ingredient; 0.01-0.3 wt. % of a surfactant; 4-6 wt. % of high molecular weight fish gelatin; and a structure former; and freeze-drying the dosed pharmaceutical formulation to form the dosage form.

In some embodiments, a dosage form includes 1.34-44.44 wt. % an active pharmaceutical ingredient; 0.13-1.33 wt. % of a surfactant; 26.67-53.62 wt. % of high molecular weight fish gelatin; 22.22-40.21 wt. % a structure former; 0.67-1.33 wt. % a pH modifier; 1.78-2.68 wt. % a sweetener; and 1.34-2.22 wt. % a flavoring agent.

Additional advantages will be readily apparent to those skilled in the art from the following detailed description. The examples and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE DRAWINGS

Exemplary embodiments are described with reference to the accompanying figures, in which:

FIG. 1 illustrates a flow chart for producing a pharmaceutical dosage form disclosed herein.

FIG. 2 illustrates an identification classification system for determining when the flow of the pharmaceutical formulation in a preformed mold is acceptable or unacceptable.

FIG. 3 includes images of misshaped dosage forms containing high molecular weight fish gelatin.

### DETAILED DESCRIPTION

Disclosed herein are dosing formulations and subsequent dosage forms prepared from the dosing formulations comprising a small amount of a suitable surfactant that can reduce the surface tension of the formulation and allow it to freely flow into the preformed mold and cover the bottom most surface or base of the preformed mold resulting in a well-shaped dosage form. In addition, at these small amounts, the surfactant can be tasteless so as not to affect the taste of the finished dosage form prepared from the dosing formulation.

Accordingly, Applicants have discovered that the addition of a small amount of a suitable surfactant (e.g., poloxamer 188, sodium lauryl sulfate, docusate sodium) to the pharmaceutical formulation can reduce the surface tension of the formulation and allow it to freely flow into the preformed mold and cover the bottom most surface or base of the preformed mold when dosed. This can result in a well-shaped dosage form.

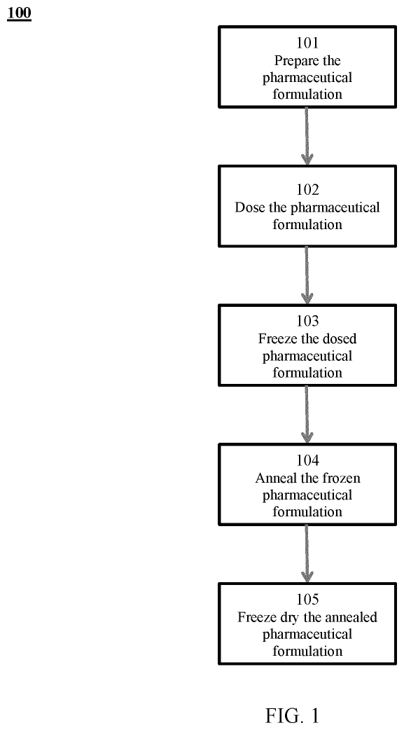

FIG. 1 illustrates a flow chart for a method 100 of producing a dosage form disclosed herein. The dosage forms (e.g., buccal/sublingual tablet, oral tablet or capsules, vaginal inserts, etc.) can dissolve for the delivery of an active pharmaceutical ingredient (“API”). At step 101, a pharmaceutical formulation can be prepared. The pharmaceutical formulation can later be dosed into the preformed mold at step 102.

The pharmaceutical formulations disclosed herein can include a matrix former such as fish gelatin. Specifically, the fish gelatin can be high molecular weight fish gelatin, standard molecular weight fish gelatin, or combinations thereof. High molecular weight fish gelatin is defined as a fish gelatin in which more than 50% of the molecular weight distribution is greater than 30,000 Daltons. Standard molecular weight fish gelatin is defined as fish gelatin in which more than 50% of the molecular weight distribution is below 30,000 Daltons. In some embodiments, the pharmaceutical formulation can include, without limitation, other gelatin, starch, or combinations thereof. Additional matrix formers can be found in EP 2624815 B 1, which is herein incorporated by reference in its entirety. The other gelatin can be bovine gelatin, porcine gelatin, or combination thereof. In some embodiments, the amount of high molecular weight fish gelatin in the pharmaceutical formulation (prior to freeze drying) can be about 2-8% w/w, 3-7% w/w, or 4-6% w/w. Unless otherwise stated herein, % w/w refers to the formulation prior to freeze drying. In some embodiments, the amount of high molecular weight fish gelatin in the pharmaceutical formulation can be less than or equal to 8% w/w, less than or equal to 7% w/w, less than or equal to 6% w/w, less than or equal to 5 w/w, less than or equal to 4% w/w, or less than or equal to 3% w/w. In some embodiments, the amount of high molecular weight fish gelatin in the pharmaceutical formulation can be more than or equal to 2% w/w, more than or equal to 3% w/w, more than or equal to 4% w/w, more than or equal to 5% w/w, more than or equal to 6% w/w, or more than or equal to 7% w/w.

The pharmaceutical formulation can also include a structure former. Suitable structure formers can include sugars including, but not limited to, mannitol, dextrose, lactose, galactose, cyclodextrin, or combinations thereof. The structure former can be used in freeze drying as a bulking agent as it crystalizes to provide structural robustness to the freeze-dried dosage form. In some embodiments, the amount of structure former in the pharmaceutical formulation can be about 1-8% w/w, 2-6% w/w, 3-6% w/w, 3-5.5% w/w, 3-5% w/w, or 3.3-5% w/w. In some embodiments, the amount of structure former in the pharmaceutical formulation can be less than or equal to 8% w/w, less than or equal to 7% w/w, less than or equal to 6% w/w, less than or equal to 5% w/w, less than or equal to 4% w/w, less than or equal to 3.3% w/w, less than or equal to 3% w/w, or less than or equal to 2% w/w. In some embodiments, the amount of structure former in the pharmaceutical formulation can be more than or equal to 1% w/w, more than or equal to 2% w/w, more than or equal to 3% w/w, more than or equal to 3.3% w/w, more than or equal to 4% w/w, more than or equal to 5% w/w, more than or equal to 6% w/w, or more than or equal to 7% w/w.

The pharmaceutical formulation may also contain an active pharmaceutical ingredient. As used herein, “active pharmaceutical ingredient” or “API” refers to a drug product that may be used in the diagnosis, cure, mitigation, treatment, or prevention of disease. Any API may be used for purposes of the present disclosure. Suitable APIs include, without limitation: analgesics and anti-inflammatory agents, antacids, anthelmintics, anti-arrhythnic agents, anti-bacterial agents, anti-coagulants, anti-depressants, anti-diabetics, anti-diarrheals, anti-epileptics, anti-fungal agents, anti-gout agents, antihypertensive agents, anti-malarials, anti-migraine agents, anti-muscarinic agents, anti-neoplastic agents and immunosuppressants, anti-protazoal agents, antirheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, and stimulants; and combinations thereof. A list of specific examples of these API may be found in U.S. Pat. No. 6,709,669, which is incorporated herein by reference. When present, the API is present in the pharmaceutical formulation in an amount that is necessary to exhibit the required physiological effect as established by clinical studies. In some embodiments, the amount of API in the pharmaceutical formulation can be about 0.05-30% w/w, 0.1-25% w/w, 2-25% w/w, 5-25% w/w, or 10-15% w/w. In some embodiments, the amount of API in the pharmaceutical formulation can be about 0.05-5% w/w, 0.1-3% w/w, or 0.2-2% w/w. In some embodiments, the amount of API in the pharmaceutical suspension can be about 0.1-10% w/w. In some embodiments, the amount of API in the pharmaceutical composition can be less than or equal to 30% w/w, less than or equal to 25 w/w, less than or equal to 20% w/w, less than or equal to 15% w/w, less than or equal to 10% w/w, less than or equal to 5% w/w, less than or equal to 2% w/w, or less than or equal to 2% w/w. In some embodiments, the amount of API in the pharmaceutical composition can be more than or equal to 0.05% w/w, more than or equal to 0.1% w/w, more than or equal to 1% w/w, more than or equal to 2% w/w, more than or equal to 5% w/w, more than or equal to 10% w/w, more than or equal to 15 w/w, more than or equal to 20% w/w, or more than or equal to 25% w/w. In some embodiments, a person of ordinary skill in the art can readily determine an appropriate amount of API to include in the dosage form or pharmaceutical composition made according to the present disclosure. In some embodiments, the API can be desmopressin and/or glycopyrrolate.

Furthermore, the pharmaceutical formulations disclosed herein include a surfactant. In some embodiments, the surfactant can be a non-ionic surfactant. In some embodiments, the non-ionic surfactant can include a polyoxyethylene-polyoxypropylene copolymer. In some embodiments, the surfactant comprises poloxamer 188 (e.g., Kolliphor® P188 by BASF) which is a non-ionic surfactant. In some embodiments, the surfactant may comprise sodium lauryl sulfate (anionic) and/or docusate sodium (anionic). Applicants discovered that the inclusion of a small amount of surfactant in the pharmaceutical formulation improves the flow characteristics of the pharmaceutical formulation during dosing. Specifically, Applicants discovered that the amount of surfactant in the pharmaceutical formulation can be about 0.001-0.5% w/w, about 0.01-0.3% w/w, or about 0.02-0.2% w/w. In some embodiments, the amount of surfactant in the pharmaceutical formulation may be less than or equal to 0.5% w/w, less than or equal to 0.4% w/w, less than or equal to 0.3% w/w, less than or equal to 0.2% w/w, less than or equal to 0.1% w/w, less than or equal to 0.05 w/w, less than or equal to 0.02% w/w, less than or equal to 0.01% w/w, or less than or equal to 0.005% w/w. In some embodiments, the amount of surfactant in the pharmaceutical formulation may be more than 0.001% w/w, more than 0.005% w/w, more than 0.01% w/w, more than 0.02% w/w, more than 0.05% w/w, more than 0.1% w/w, more than 0.2% w/w, more than 0.3% w/w, or more than 0.4% w/w. In some embodiments, as the amount of surfactant in a pharmaceutical formulation increases, the surface tension will also decrease. However, at a certain point, the surface tension may no longer decrease (i.e., the surface tension may plateau as the amount of surfactant increases, and/or may increase slightly); once this point is reached, additional surfactant may not have a positive effect on the surface tension of the pharmaceutical formulation.

The pharmaceutical formulation may also contain additional pharmaceutically acceptable agents or excipients. Such additional pharmaceutically acceptable agents or excipients include, without limitation, sugars, such as mannitol, dextrose, and lactose, inorganic salts, such as sodium chloride and aluminum silicates, gelatins of mammalian origin, fish gelatin, modified starches, preservatives, antioxidants, viscosity enhancers, coloring agents, flavoring agents, pH modifiers, sweeteners, taste-masking agents, and combinations thereof. Suitable coloring agents can include red, black and yellow iron oxides and FD & C dyes such as PD & C Blue No. 2 and FD & C Red No. 40, and combinations thereof. Suitable flavoring agents can include mint, raspberry, licorice, orange, lemon, grapefruit, caramel, vanilla, cherry (e.g., black cherry), and grape flavors and combinations of these. In some embodiments, the pharmaceutical formulation can include at least one flavoring agent in an amount of 0.1-5% w/w, 0.1-1% w/w, 0.25-0.75% w/w, 0.4-0.6% w/w, or 0.5% w/w. In some embodiments, the pharmaceutical formulation can include at least one flavoring agent in an amount of 0.1-0.5 w/w. In some embodiments, the amount of flavoring agent in the pharmaceutical formulation can be at least 0.1% w/w, at least 0.2% w/w, at least 0.3% w/w, at least 0.4% w/w, at least 0.5% w/w. In some embodiments, the amount of flavoring agent in the pharmaceutical formulation can be at most 0.5 w/w, at most 0.4% w/w, at most 0.3% w/w, or at most 0.2% w/w.

Suitable pH modifiers can include citric acid, tartaric acid, phosphoric acid, hydrochloric acid, maleic acid, sodium hydroxide (e.g., 3% w/w sodium hydroxide solution), and combinations thereof. In some embodiments, the pharmaceutical formulation has an amount of a pH modifier (i.e., Q.S. target pH) to maintain a target pH of about 4-6, about 4.5-5.5, about 4.7-5.3, about 4.7-5, or about 4.8-4.9. In some embodiments, the pharmaceutical formulation can include 0.05-0.3% w/w pH modifier. In some embodiments, the pharmaceutical formulation can include at least 0.05% w/w, at least 0.1% w/w, at least 0.15% w/w, at least 0.2% w/w, at least 0.25% w/w, or at least 0.3% w/w pH modifier. In some embodiments, the pharmaceutical formulation can include at most 0.3% w/w, at most 0.25% w/w, at most 0.2% w/w, at most 0.15% w/w, or at most 0.1% w/w pH modifier.

Suitable sweeteners can include sucralose, aspartame, acesulfame K and thaumatin, and combinations thereof. In some embodiments, the pharmaceutical formulation can include at least one sweetener in an amount of 0.1-1% w/w, 0.2-0.5% w/w, 0.2-0.4% w/w, 0.3-0.4% w/w, or 0.35% w/w. In some embodiments, the pharmaceutical formulation can include at least one sweetener in an amount of at least 0.2% w/w, at least 0.25% w/w, at least 0.3% w/w, or at least 0.35% w/w. In some embodiments, the pharmaceutical formulation can include at least one sweetener in an amount of at most 0.4% w/w, at most % w/w, at most 0.3% w/w, or at most 0.25% w/w.

Suitable taste-masking agents can include sodium bicarbonate, ion-exchange resins, cyclodextrin inclusion compounds, adsorbates or microencapsulated actives, and combinations thereof. One of ordinary skill in the art can readily determine suitable amounts of these various additional excipients if desired.

The pharmaceutical formulation can also include a solvent. In some embodiments, the solvent can be ethanol, isopropanol, other lower alkanols, water (e.g., purified water), or combinations thereof. In some embodiments, the balance remaining of the pharmaceutical formulation is the solvent (i.e., Q.S. 100%). In some embodiments, the pharmaceutical formulation can include 77.5-92.54% w/w solvent.

In some embodiments, the pharmaceutical formulation can also include a muco-adhesive such as gum. Suitable gums include, but are not limited to, acacia, guar, agar, xanthan, gellan, carageenan, curdlan, konjac, locust bean, welan, gum tragacanth, gum arabic, gum karaya, gum ghatti, pectins, dextran, glucomannan, and alginates, or combinations thereof.

As stated above, the pharmaceutical formulation is prepared in step 101. The pharmaceutical formulations can be prepared by any conventional method. In some embodiments, a premix of the pharmaceutical formulation can be formed by dissolving the matrix former, the structure former, and the surfactant in the solvent. For example, high molecular weight fish gelatin, mannitol, and poloxamer 188 can be dissolved in water. The premix can be stirred and/or heated to about 40-80° C., about 50-70° C., about 55-65° C., or about and maintained for about 10-60 minutes.

Once the matrix former, structure former, and surfactant are fully dissolved, the premix can be cooled to about 15-30° C., 20-30° C., about 20-25° C., or about 21-25° C. After cooling, the API can be added to the premix and allowed to dissolve or disperse to form a uniform suspension. Subsequently, the pH can be adjusted to about 4-10, 4-6, about 4.5-5.5, about 4.7-5.3, about 4.7-5, or about 4.8-4.9 using a pH modifier. For example, the pH can be adjusted to 4.8-4.9 with citric acid powder. In some embodiments, the pH can be adjusted with any of the pharmaceutical acceptable acids such as citric acid, maleic acid, tartaric acid or hydrochloric acid. In some embodiments, the pH can be about 7-10 and the pH modifier can be alkali metal hydroxides, alkaline eaul metal hydroxides, or mixtures thereof. Examples of alkali metal hydroxides include sodium hydroxide, potassium hydroxide and mixtures thereof. One example of an alkaline earth metal hydroxide is magnesium hydroxide.

This mixture can be made up to a desired batch size with solvent (i.e., the pharmaceutical formulation). For example, an amount of water can be added to the mixture, as necessary, to Q.S. to 100%.

Pharmaceutical formulations provided herein can be characterized by properties including, for example, surface tension, viscosity, and relative density. Surface tension, for example, may be decreased with the presence of surfactant in pharmaceutical compositions provided herein. Pharmaceutical formulations with a surface tension that is too high can increase the occurrence of wedging or otherwise misshapen dosage forms. Specifically, the surface tension in pharmaceutical compositions that do not comprise any surfactant may be 70-100 mN/m. However, the surface tension of pharmaceutical formulations comprising surfactant may be 50-80 mN/m, 60-80 mN/m, or 60-70 mN/m. In some embodiments, the surface tension of pharmaceutical formulations comprising surfactant may be less than or equal to 80 mN/m, less than or equal to 70 mN/m, less than or equal to 60 mN/m, less than or equal to 55 mN/m, less than or equal to 50 mN/m, less than or equal to 45 mN/m, less than or equal to 40 mN/m, or less than or equal to 35 mN/m. In some embodiments, the surface tension of pharmaceutical formulations comprising surfactant may be more than 30 mN/m, more than 40 mN/m, more than 45 mN/mmore than 50 mN/m, more than 60 mN/m, or more than 70 mN/m. In some embodiments, the surface tension of pharmaceutical formulations comprising surfactant may be 2-50%, 10-30%, or 10-20% less than the surface tension of pharmaceutical formulations without surfactant. In some embodiments, the surface tension of pharmaceutical formulations comprising surfactant may be less than or equal to 50%, less than or equal to 40%, less than or equal to 30%, less than or equal to 20%, less than or equal to 10% , or less than or equal to 5% less than the surface tension of pharmaceutical formulations without surfactant. In some embodiments, the surface tension of pharmaceutical formulations comprising surfactant may be more than 2%, more than 5%, more than 10%, more than 20%, more than 30%, or more than 40% less than the surface tension of pharmaceutical formulations without surfactant.

Pharmaceutical formulations provided herein may also be characterized by their viscosity. Pharmaceutical formulations having too high of a viscosity may cause wedging or otherwise misshapen dosage forms. Pharmaceutical formulations having too low of a viscosity may be more difficult to dose into blister packs accurately. Pharmaceutical formulations comprising surfactant may have a viscosity of 5-15 mPa s, 7-13 mPa s, or 9-12 mPa s. In some embodiments, the pharmaceutical formulations comprising surfactant may have a viscosity of less than or equal to 15 mPa s, less than or equal to 14 mPa s, less than or equal to 13 mPa s, less than or equal to 12 mPa s, less than or equal to 11 mPa s, less than or equal to 10 mPa s, less than or equal to 9 mPa s, less than or equal to 8 mPa s, less than or equal to 7 mPa s, or less than or equal to 6 mPa s. In some embodiments, the pharmaceutical formulations comprising surfactant may have a viscosity of more than or equal to 5 mPa s, more than or equal to 6 mPa s, more than or equal to 7 mPa s, more than or equal to 8 mPa s, more than or equal to 9 mPa s, more than or equal to 10 mPa s, more than or equal to 11 mPa s, more than or equal to 12 mPa s, more than or equal to 13 mPa s, or more than or equal to 14 mPa s. In some embodiments, the presence of surfactant in the pharmaceutical formulations provided herein may have little, if any, effect on the viscosity.

Pharmaceutical formulations provided herein may also be characterized by their relative density. In some embodiments, the presence of surfactant in a pharmaceutical formulation provided herein may decrease the relative density of the pharmaceutical formulation (i.e., such that it is lower than that of a pharmaceutical formulation without surfactant). In some embodiments, the presence of surfactant in a pharmaceutical formulation provided herein may not have any impact on the relative density of the pharmaceutical formulation (i.e., as compared to that of a pharmaceutical formulation without surfactant). In some embodiments, the relative density of a pharmaceutical formulation provided herein may be 1.0-1.4 or 1.2-1.3. In some embodiments, the relative density of a pharmaceutical formulation provided herein may be less than or equal to 1.4, less than or equal to 1.3, less than or equal to 1.2, or less than or equal to 1.1. In some embodiments, the relative density of a pharmaceutical provided herein may be more than or equal to 1.0, more than or equal to 1.1, more than or equal to 1.2, or more than or equal to 1.3.

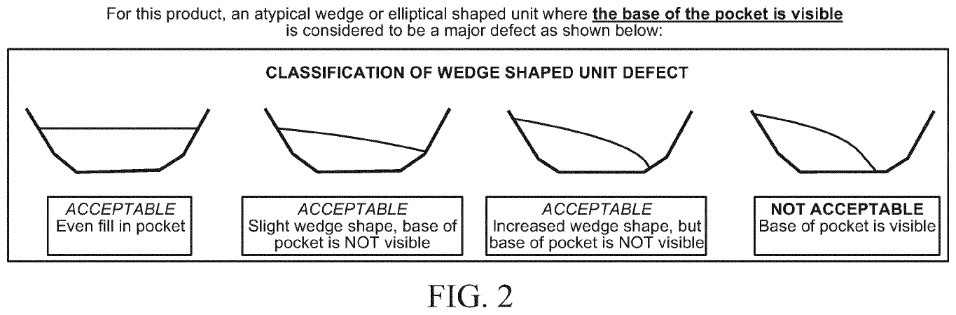

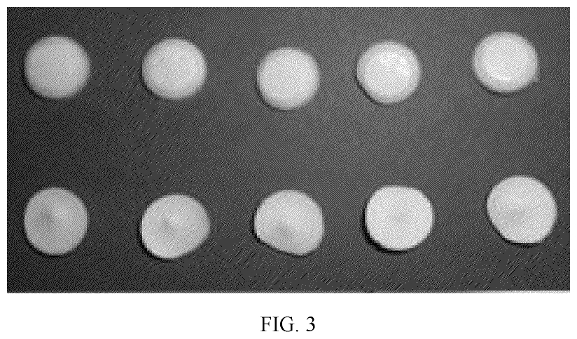

Additionally, dosage forms prepared with pharmaceutical formulations provided herein may be characterized by the occurrence of wedging, or misshapen dosage forms. (FIGS. 2 and 3, discussed in further detail below, provide images of wedging and misshapen dosage forms). In some embodiments, the presence of surfactant in the pharmaceutical formulation can minimize the occurrence of both minor and major wedge shaped dosage forms. A minor wedge shaped dosage form can include an inclined upper surface (i.e., instead of a horizontal upper surface). Two examples of minor wedge shaped dosage forms are shown in FIG. 2. A major wedge shaped dosage form can occur when the pharmaceutical formulation, when dosed into a blister pocket, clings to a side of the blister pocket so much so that the dose does not completely fill the base of the blister pocket. FIG. 2 shows an example of a major wedge shaped dosage form.

In some embodiments, the presence of surfactant (e.g., poloxamer, sodium lauryl sulfate, docusate sodium) in the pharmaceutical formulation can decrease the occurrence of minor wedge shaped dosage forms by 30-100%. In some embodiments, the presence of surfactant in the pharmaceutical formulation can decrease the occurrence of minor wedge shaped dosage forms by less than or equal to 100%, less than or equal to 90%, less than or equal to 80%, less than or equal to 70%, less than or equal to 60%, less than or equal to 50%, or less than or equal to 40%. In some embodiments, the presence of surfactant in the pharmaceutical formulation can decrease the occurrence of minor wedge shaped dosage forms by more than or equal to 30%, more than or equal to 40%, more than or equal to 50%, more than or equal to 60%, more than or equal to 70%, more than or equal to 80%, or more than or equal to 90%. In some embodiments, the presence of surfactant in the pharmaceutical formulation can decrease the occurrence of major wedge shaped dosage forms by 70-100%. In some embodiments, the presence of surfactant in the pharmaceutical formulation can decrease the occurrence of major wedge shaped dosage forms by 50-100%. In some embodiments, the presence of surfactant in the pharmaceutical formulation can decrease the occurrence of major wedge shaped dosage forms by less than or equal to 100%, less than or equal to 90%, less than or equal to 80%, less than or equal to 70%, or less than or equal to 60%. In some embodiments, the presence of surfactant in the pharmaceutical formulation can decrease the occurrence of major wedge shaped dosage units by more than or equal to 50%, more than or equal to 60%, more than or equal to 70%, more than or equal to 80%, or more than or equal to 90%.

At step 102 of FIG. 1, the pharmaceutical formulation can be dosed into a preformed mold. In some embodiments of the present disclosure, the preformed mold is a blister pack with one or more blister pockets. Predetermined aliquots in an amount of less than about 300 mg, less than about 250 mg, less than about 225 mg, or less than about 200mg wet filling dosing weight of the pharmaceutical formulation can be metered into preformed molds. In some embodiments, the formulation can be dosed at about 10-25° C. In some embodiments, the preformed molds are aluminum blister trays. Other suitable blister trays can include blister packaging material with a PVC product contact layer.

To achieve a satisfactory dosage form that is not misshapen, the base of each blister pocket should be completely covered. (See FIG. 2, which provides a diagram of a dosage form in a blister pocket exhibiting major wedging since it does not completely cover the base of the blister pocket). When the base of the blister pocket is not completely covered, the dosage form can have major wedging characteristics. Additionally, to minimize the occurrence of minor wedging, the pharmaceutical formulation can be dosed into each blister pocket such that any incline in the upper surface of the dosage (which can be caused when the pharmaceutical formulation clings/adheres to one side of the blister pocket more so than another side) is minimized (See FIG. 2, which provides two examples of dosage forms that have minor wedging). Ideally, the pharmaceutical formulation can be dosed such that no wedging occurs.

At step 103 of FIG. 1, the dosed pharmaceutical formulations can then be frozen in the preformed molds. The dosed pharmaceutical formulations in the preformed molds can be frozen by any means known in the art. For example, the formulations can be passed through a cryogenic chamber (e.g., liquid nitrogen tunnel). The temperature during freezing can be between about −40 to −90° C., about −50 to −70° C., about −55 to −65° C., or about -60° C. The freezing duration can range from about 1.5-5 minutes, about 2-4.5 minutes, about 2.5-4 minutes, about 3-4 minutes, about 3-3.5 minutes, or about 3.25 minutes. For example, the dosed pharmaceutical formulation can be frozen at −60° C. for 3 minutes and 15 seconds.

At step 104 of FIG. 1, the frozen units in the preformed molds can be collected in placed in a freezer at a temperature of about −25° C. and annealed (i.e., frozen hold) for a period of time to crystallize the structure former. Structure former crystallization can provide the frozen units with the structural strength to prevent the collapse of the frozen units during freeze drying. The annealing time can range from about 0.25-3 hours, about 0.5-2 hours, about 0.75-1.25 hours, or about 1 hour.

After annealing, the annealed frozen units can be freeze-dried in step 105 to form the dosage form. During the freeze-drying process, the water is sublimated from the frozen units. In some embodiments, the frozen units can be loaded onto the shelves of a freeze-drier. Once the annealed frozen units are in the freeze-drier, the freeze-drying cycle can be initiated. In some embodiments, a vacuum can be pulled and the shelf temperature raised once the freeze-drying cycle is initiated. The freeze-drier can operate at low pressure (i.e., vacuum). In some embodiments, the freeze-drier can operate at a pressure of about less than or equal to 1000 mbar, about less than or equal to 900 mbar, about less than or equal to 800 mbar, about less than or equal to 700 mbar, about less than or equal to 600 mbar, about less than or equal to 500 mbar, or about less than or equal to 400 mbar. The drying temperature can be about 10° C. to about −10° C., about 5° C. to about −5° C., or about 0° C. In addition, the drying time can be about 2-20 hours, about 4-17 hours, or about 5-16 hours. After freeze drying, the freeze-dried dosage forms can be removed from the freeze-drier and inspected for any defects (quality inspection as described below).

The dosage forms described herein can include at least one API, at least one surfactant, at least one matrix former (e.g., HMW fish gelatin, at least one structure former, at least one pH modifier, at least one sweetener, and/or at least one flavoring agent. In some embodiments, the dosage form can include the API in an amount of 1.34-44.44% w/w. In some embodiments, the dosage form can include the API in an amount of at least 1.34% w/w, at least 2% w/w, at least 5% w/w, at least 10% w/w, at least 15% w/w, at least 20% w/w, at least 25% w/w, at least 30% w/w, at least 35% w/w, or at least 40% w/w. In some embodiments, the dosage form can include the API in an amount of at most 44.44% w/w, at most 40% w/w, at most 35% w/w, at most 30% w/w, at most 25% w/w, at most 20% w/w, at most 15% w/w, at most 10% w/w, at most 5% w/w, or at most 2% w/w.

In some embodiments, the dosage form can include at least one surfactant in an amount of 0.13-1.33% w/w. In some embodiments, the dosage form can include at least one surfactant in an amount of at least 0.13% w/w, at least 0.2% w/w, at least 0.3% w/w, at least 0.4% w/w, at least 0.5% w/w, at least 0.6% w/w, at least 0.7% w/w, at least 0.8% w/w, at least 0.9% w/w, at least 1% w/w, at least 1.1% w/w, at least 1.2% w/w, or at least 1.3% w/w. In some embodiments, the dosage form can include at least one surfactant in an amount of at most 1.33% w/w, at most 1.3% w/w, at most 1.2% w/w, at most 1.1% w/w, at most 1% w/w, at most 0.9% w/w, at most 0.8% w/w, at most 0.7% w/w, at most 0.6% w/w, at most 0.5% w/w, at most 0.4% w/w, at most 0.3% w/w, or at most 0.2% w/w.

In some embodiments, the dosage form can include at least one matrix former (e.g., HMW fish gelatin) in an amount of 26.67-53.62% w/w. In some embodiments, the dosage form can include at least one matrix former in an amount of at least 26.67% w/w, at least 30% w/w, at least 35% w/w, at least 40% w/w, at least 45% w/w, or at least 50% w/w. In some embodiments, the dosage form can include at least one matrix former in an amount of at most 53.62% w/w, at most 50% w/w, at most 45% w/w, at most 40% w/w, at most 35% w/w, or at most 30% w/w.

In some embodiments, the dosage form can include at least one structure former in an amount of 22.22-40.21% w/w. In some embodiments, the dosage form can include at least one structure former in an amount of at least 22.22% w/w, at least 25% w/w, at least 30% w/w, at least 35% w/w, or at least 40% w/w. In some embodiments, the dosage form can include at least one structure former in an amount of at most 40.21% w/w, at most 40% w/w, at most 35% w/w, at most 30% w/w, or at most 25% w/w.

In some embodiments, the dosage form can include at least on pH modifier in an amount of 0.67-1.33% w/w. In some embodiments, the dosage form can include at least on pH modifier in an amount of at least 0.67% w/w, at least 0.7% w/w, at least 0.8% w/w, at least 0.9% w/w, at least 1% w/w, at least 1.1% w/w, at least 1.2% w/w, or at least 1.3% w/w. In some embodiments, the dosage form can include at least one pH modifier in an amount of at most 1.33% w/w, at most 1.3% w/w, at most 1.2% w/w, at most 1.1% w/w, at most 1% w/w, at most 0.9% w/w, at most 0.8% w/w, or at most 0.7% w/w.

In some embodiments, the dosage form can include at least one sweetener in an amount of 1.78-2.68% w/w. In some embodiments, the dosage form can include at least one sweetener in an amount of at least 1.78% w/w, at least 1.8% w/w, at least 1.9% w/w, at least 2% w/w, at least 2.1% w/w, at least 2.2% w/w, at least 2.3% w/w, at least 2.4% w/w, at least 2.5% w/w, or at least 2.6% w/w. In some embodiments, the dosage form can include at least one sweetener in an amount of at most 2.68% w/w, at most 2.6% w/w, at most 2.5% w/w, at most 2.4% w/w, at most 2.3% w/w, at most 2.2% w/w, at most 2.1% w/w, at most 2% w/w, at most 1.9% w/w, or at most 1.8% w/w.

In some embodiments, the dosage form can include at least one flavoring agent in an amount of 1.34-2.22% w/w. In some embodiments, the dosage form can include at least one flavoring agent in an amount of at least 1.34% w/w, at least 1.4% w/w, at least 1.5% w/w, at least 1.6% w/w, at least 1.7% w/w, at least 1.8% w/w, at least 1.9% w/w, at least 2% w/w, at least 2.1% w/w, or at least 2.2% w/w. In some embodiments, the dosage form can include at least one flavoring agent in an amount of at most 2.22% w/w, at most 2.2% w/w, at most 2.1% w/w, at most 2% w/w, at most 1.9% w/w, at most 1.8% w/w, at most 1.7% w/w, at most 1.6% w/w, at most 1.5% w/w, or at most 1.4% w/w.

The dosage forms of the present disclosure are dissolving dosage forms and accordingly have the distinct advantage of a faster disintegrating time. The route of administration may be oral, vaginal or nasal, though preferably oral. Once placed in the oral cavity and in contact with saliva, a dosage form can disintegrate within about 1 to about 180 seconds, about 1 to about 120 seconds, about 1 to about 60 seconds, preferably within about 1 to about 30 seconds, more preferably within about 1 to about 10 seconds and most preferably in less than about 5 seconds.

### EXAMPLES

Example 1: In order to determine if a 0.1% w/w concentration of surfactant was suitable for inclusion in the pharmaceutical formulations, a series of 8 bench scale batches were prepared. Four of these batches were placebo and four of these contained desmopressin as the API at a concentration to give a dose of 480 μg. Each batch contained concentrations of poloxamer 188 at 0, 0.05, 0.1, or 0.2% w/w in the pharmaceutical formulation. At these low concentrations, the poloxamer is considered to be tasteless. By following this approach, it could be determined whether formulations giving doses of desmopressin from 480 μg down to placebo are improved in terms of their flow characteristics when a large window of poloxamer concentration is used.

Each formulation was dosed into five layer foil pack with preformed molds/blister pockets designed to be filled with aliquots of up to 300 mg (fill weight) of the formulation. To determine the outcome using the two most likely scenarios in terms of fill weight dosed, each formulation would be dosed as a 200 mg fill and a 250 mg fill. The following Table 1 provides the details of each formulation used.

The batches were prepared by adding the gelatin, mannitol, and poloxamer (where applicable) to the bulk (80%) of purified water and heating to 60° C. while stirring with a magnetic follower. Once the gelatin had fully dissolved the solutions were cooled to 23° C. (±2° C.) at which point the drug was added to the applicable solutions and allowed to dissolve. Where placebo formulations are referenced, no drug is added. The pH of each solution was then adjusted to 4.8-4.9 with citric acid powder. Finally, purified water was added to make each batch up to 100%.

A Hamilton Microlab was used to dose either 250 mg (labelled suffix A) or 200 mg (labelled suffix B) in to 5 layer foil blister trays with preformed mold. The dosed pharmaceutical formulation was then frozen and then freeze dried. After dosing, the flow of the pharmaceutical formulations were inspected. Specifically, an atypical wedge or elliptical shaped unit in the preformed mold where the base or bottom most surface of the preformed mold is visible is considered to be a major defect as shown in FIG. 2.

The dosed trays were then frozen in a freeze tunnel set at −60° C. and a residence time of 3 minutes and 15 seconds and then transferred to a Refrigerated Freezer Cabinet (“RFC”) where it was held for approximately 1 hour prior to freeze drying. A drying temperature of 0° C. was used and the product was dried for 16 hours although the drying trace showed that the product was dry in approximately 5 hours.

TABLE 1

| | | Solution Flow | |
|---|---|---|---|
| BATCH | Formulation Composition | A - 250 mg fill | B - 200 mg fill |
| 1 | 0.05% w/w poloxamer 188 | Acceptable | Acceptable |
| | 5.0% w/w HMW fish gelatin | appearance | appearance |
| | 4.1% w/w mannitol | | |
| | Q.S. pH 4.8-4.9 (citric acid) | | |
| | Q.S. 100% water | | |
| 2 | 0.1% w/w poloxamer 188 | Acceptable | Acceptable |
| | 5.0% w/w HMW fish gelatin | appearance | appearance |
| | 4.1% w/w mannitol | | |
| | Q.S. pH 4.8-4.9 (citric acid) | | |
| | Q.S. 100% water | | |
| 3 | 0.2% w/w poloxamer 188 | Acceptable | Acceptable |
| | 5.0% w/w HMW fish gelatin | appearance | appearance |
| | 4.1% w/w mannitol | | |
| | Q.S. pH 4.8-4.9 (citric acid) | | |
| | Q.S. 100% water | | |
| 4 | No poloxamer 188 | Poor solution flow | Poor solution flow |
| | 5.0% w/w HMW fish gelatin | Acceptable | Irregular, |
| | 4.1% w/w mannitol | Appearance | noncylindrical units |
| | Q.S. pH 4.8-4.9 (citric acid) | | with poor appearance |
| | Q.S. 100% water | | |
| 5 | 0.22% w/w desmopressin | Acceptable | Acceptable |
| | 0.05% w/w poloxamer 188 | appearance | appearance |
| | 5.0% w/w HMW fish gelatin | | |
| | 4.1% w/w mannitol | | |
| | Q.S. pH 4.8-4.9 (citric acid) | | |
| | Q.S. 100% water | | |
| 6 | 0.22% w/w desmopressin | Acceptable | Acceptable |
| | 0.1% w/w poloxamer 188 | appearance | appearance |
| | 5.0% w/w HMW fish gelatin | | |
| | 4.1% w/w mannitol | | |
| | Q.S. pH 4.8-4.9 (citric acid) | | |
| | Q.S. 100% water | | |
| 7 | 0.22% w/w desmopressin | Acceptable | Acceptable |
| | 0.2% w/w poloxamer 188 | appearance | appearance |
| | 5.0% w/w HMW fish gelatin | | |
| | 4.1% w/w mannitol | | |
| | Q.S. pH 4.8-4.9 (citric acid) | | |
| | Q.S. 100% water | | |
| 8 | 0.22% w/w desmopressin | Poor solution flow | Poor solution flow |
| | No poloxamer 188 | Acceptable | Irregular, |
| | 5.0% w/w HMW fish gelatin | Appearance | noncylindrical units |
| | 4.1% w/w mannitol | | with poor appearance |
| | Q.S. pH 4.8-4.9 (citric acid) | | |
| | Q.S. 100% water | | |

The results in Table 1 above indicate that the inclusion of poloxamer 188 between 0.05% w/w and 0.2% w/w is effective at improving the flow characteristics of the placebo and desmopressin pharmaceutical formulations. The only formulations to give unsatisfactory units were those that did not contain poloxamer 188 and were dosed as a 200 mg fill. However, when the non poloxamer 188 formulations were dosed as a 250 mg fill, the flow was reported to be slow albeit sufficiently rapid to form acceptable shaped units.

Example 2: The impact of poloxamer 188 on the flow properties of pharmaceutical formulations described herein when dosed into blister packs was investigated. In particular, the pharmaceutical formulations tested herein were dosed into blister packs as provided herein to minimizing wedging/misshapen appearance of the final dosage forms.

Each formulation was dosed into five layer foil pack with preformed molds/blister pockets designed to be filled with aliquots of up to 250 mg (fill weight) of the formulation. each formulation was dosed as a 150 mg fill.

The batches were prepared by adding the gelatin, mannitol, and poloxamer (where applicable) to the purified water and heating to 60° C. while stirring with a magnetic follower. Once the gelatin had fully dissolved the solutions were cooled to 20° C. (±2° C.). For these placebo formulations no drug was added.

A Hibar dosing pump was used to dose 150 mg in to 5 layer foil blister with preformed mold. The dosed pharmaceutical formulation was then frozen and then freeze dried.

The dosed trays were then frozen in a freeze tunnel set at −70° C. and a residence time of 3 minutes and 15 seconds and then transferred to a Refrigerated Freezer Cabinet (“RFC”) where it was held prior to freeze drying. A drying temperature of 0° C. was used and the product was dried for 6 hours.

Table 2, below, provides the five different pharmaceutical formulations (each pharmaceutical formulation is represented by a Batch number) that were tested.

TABLE 2

| | (% w/w) | | (% w/w) | |
|---|---|---|---|---|
| | HMW Fish | (% w/w) | Poloxamer | |
| Batch | Gelatin | Mannitol | 188 | Rationale |
| 1 | 5.00 | 4.10 | 0.00 | Evaluate the basic |
| | | | | pharmaceutical |
| | | | | formulation without |
| | | | | poloxamer 188 |
| 2 | | | 0.02 | Evaluate 4 different |
| 3 | | | 0.05 | concentrations of |
| 4 | | | 0.10 | poloxamer 188 to |
| 5 | | | 0.20 | determine optimum |
| | | | | level with respect to |
| | | | | reducing |
| | | | | wedging/misshaped |
| | | | | units |

Each of the pharmaceutical formulations provided in Table 2 were tested for viscosity, density, pH, and surface tension. The dried tablets (i.e., dosage forms) were visually inspected for the presence of wedging (i.e., misshapen units). Table 3, below, shows the properties (i.e., pH, viscosity, relative density, and surface tension) of each pharmaceutical formulation/solution.

TABLE 3

| | | Viscosity | Relative | Surface tension |
|---|---|---|---|---|
| Batch | pH | mPa s | Density | (mN/m) |
| 1 | 6.54 | 9.3 | 1.19 | 78.41 |
| 2 | 6.54 | 9.7 | 1.21 | 70.08 |
| 3 | 6.55 | 9.5 | 1.24 | 68.40 |
| 4 | 6.54 | 9.9 | 1.24 | 63.84 |
| 5 | 6.55 | 9.6 | 1.24 | 64.42 |

As demonstrated by the data in Table 3, the poloxamer does not appear to have any effect on the pH, viscosity, or relative density of the solutions. However, it does appear that the poloxamer affects the surface tension of the solutions. As the amount of poloxamer in the pharmaceutical formulation increases from 0% w/w (i.e., Batch 1) to 0.1% w/w (i.e., Batch 4), the surface tension of the solution decreases. An additional increase in poloxamer in the pharmaceutical formulation to 0.2% w/w (i.e., Batch 5) may not show a further decrease in surface tension.

The dried tablets (i.e., dosage forms) were visually inspected for the presence of wedging (i.e., misshapen units). Table 4, below, shows the properties of the dosage forms prepared by the pharmaceutical compositions provided in Table 2.

TABLE 4

| | % w/w Poloxamer | | |
|---|---|---|---|
| | 188 in dosing mix | % minor | % major wedge |
| | prior to freeze | Wedge shaped | shaped dosage |
| Batch | drying) | dosage forms | forms |
| 1 | 0.00 | 67 | 20 |
| 2 | 0.02 | 33 | 0 |
| 3 | 0.05 | 0 | 0 |
| 4 | 0.10 | 7 | 0 |
| 5 | 0.20 | 0 | 0 |

As shown in Table 4, the presence of poloxamer 188 in dosage forms described herein can improve the flow properties of the pharmaceutical formulation into the blister pockets of the blister packs. In particular, there is a significant reduction in the occurrence/percentage of both minor and major wedge-shaped units in dosage forms comprising a poloxamer 188 concentration of 0.02% w/w. As the concentration of poloxamer 188 increases in the dosage forms, the occurrence/percentage of both minor and major wedge-shaped units can be reduced to zero. As shown, the percentage of minor wedge-shaped units is zero at concentrations of 0.05% w/w and 0.20% w/w poloxamer 188, and the percentage of major wedge-shaped units at concentrations of 0.02% w/w, 0.05 w/w, 0.10% w/w, and 0.20% w/w poloxamer 188.

Example 3: The effects of alternative surfactants (i.e., sodium laurel sulfate (SLS) and docusate sodium) on the flow properties of pharmaceutical formulations provided herein when dosed into blister packs was studied. The specific goal of this study was to observe the effects of these specific surfactants on the occurrence of wedging/misshapen dosage forms. The specific formulation of each pharmaceutical formulation that was tested is provided below in Table 5. The manufacturing method was the same as that used for example 2.

TABLE 5

| | (% w/w) | | | | |
|---|---|---|---|---|---|
| | HMW | | (% w/w) | (% w/w) | |
| | Fish | (% w/w) | Sodium | Docusate | |
| Batch | Gelatin | Mannitol | Lauryl Sulfate | Sodium | Rationale |
| 1 | 5.00 | 4.10 | 0.00 | 0.00 | Evaluate the basic |
| | | | | | pharmaceutical |
| | | | | | formulation |
| | | | | | without surfactant |
| 2 | | | — | 0.001 | Evaluate different |
| 3 | | | — | 0.01 | concentrations of |
| 4 | | | — | 0.10 | docusate sodium to |
| | | | | | determine optimum |
| | | | | | level with respect |
| | | | | | to reducing |
| | | | | | wedging/misshaped |
| | | | | | units |
| 5 | | | 0.02 | — | Evaluate different |
| 6 | | | 0.05 | — | concentrations of |
| 7 | | | 0.10 | — | sodium lauryl |
| | | | | | sulfate to determine |
| | | | | | optimum level with |
| | | | | | respect to reducing |
| | | | | | wedging/misshaped |
| | | | | | units |

Each of the pharmaceutical formulations provided in Table 5 were tested for viscosity, density, pH, and surface tension. This data is provided below in Table 6.

TABLE 6

| | | Viscosity | Relative | Surface tension |
|---|---|---|---|---|
| Batch | pH | mPa s | Density | (mN/m) |
| 1 | 6.54 | 9.3 | 1.19 | 78.41 |
| 2 | 7.02 | 10.0 | 1.20 | 76.13 |
| 3 | 7.02 | 9.4 | 1.22 | 60.20 |
| 4 | 7.12 | 11.6 | 1.22 | 35.52 |
| 5 | 7.04 | 10.8 | 1.23 | 58.25 |
| 6 | 7.09 | 11.6 | 1.22 | 57.40 |
| 7 | 7.16 | 15.5 | 1.16 | 55.81 |

As shown in Table 6, neither docusate sodium nor sodium lauryl sulfate have any significant effect on the pH, viscosity, or density of the pharmaceutical formulations in solution.

Additionally, the occurrence of wedging/misshapen dosage forms was observed in dosage forms prepared with the pharmaceutical formulations of Table 5. This data is provided below in Table 7.

TABLE 7

| | % w/w docusate | | | |
|---|---|---|---|---|
| | sodium in | % w/w sodium | | |
| | dosing mix | lauryl sulfate in | % minor wedge | % major wedge |
| | (prior to freeze | dosing mix (prior | shaped dosage | shaped dosage |
| Batch | drying) | to freeze drying) | forms | forms |
| 1 | 0.00 | 0.00 | 67 | 20 |
| 2 | 0.001 | — | 43 | 7 |
| 3 | 0.01 | — | 0 | 0 |
| 4 | 0.10 | — | 0 | 0 |
| 5 | — | 0.02 | 33 | 0 |
| 6 | — | 0.05 | 7 | 0 |
| 7 | — | 0.10 | 33 | 0 |

As indicated in Table 7, the presence of surfactants such as docusate sodium and/or sodium lauryl sulfate can improve the flow of the pharmaceutical formulations (i.e., the pharmaceutical formulations of Table 5) when dosed in the blister pockets. For example, a significant reduction in the occurrence of minor and major wedge shaped dosage forms prepared from pharmaceutical formulations comprising 0.001% w/w docusate sodium (i.e., Batch 2) as compared with dosage forms having no docusate sodium. Further, in dosage forms having a docusate sodium concentration of 0.01 and 0.10% w/w, there was no occurrence of minor or major wedge shaped dosage forms.

Similarly, there is a significant reduction in the occurrence of minor and major wedge shaped dosage forms at a sodium lauryl sulfate (SLS) concentration of 0.02% w/w as compared to dosage forms with no SLS. The occurrence of major wedge shaped dosage forms is zero in dosage forms prepared from pharmaceutical formulations comprising 0.01 and 0.10% w/w SLS. There is less of a clear trend with minor wedge shaped dosage forms prepared from pharmaceutical formulations comprising 0.01 and 0.10% w/w SLS.

Example 4: Tests were conducted on pharmaceutical formulations and dosage forms prepared with said pharmaceutical formulations comprising the API glycopyrrolate. Table 8, below, provides the specific pharmaceutical formulations that were tested.

TABLE 8

| | Batch Number | | | | | |
|---|---|---|---|---|---|---|
| | 1 | 2 | 3 | 4 | 5 | 6 |
| Material | % w/w | | | | | |
| Purified Water | 88.59 | 88.64 | 88.16 | 88.16 | 87.65 | 87.72 |
| Gelatin | 5.00 | 5.00 | 5.25 | 5.25 | 5.50 | 5.50 |
| (Fish HMW) | | | | | | |
| Mannitol | 4.00 | 4.00 | 4.20 | 4.20 | 4.40 | 4.40 |
| Poloxamer 188 | 0.10 | 0.025 | 0.05 | 0.05 | 0.10 | 0.025 |
| Glycopyrrolate | 1.33 | 1.33 | 1.33 | 1.33 | 1.33 | 1.33 |
| Sucralose | 0.35 | 0.35 | 0.35 | 0.35 | 0.35 | 0.35 |
| Black Cherry | 0.50 | 0.50 | 0.50 | 0.50 | 0.50 | 0.50 |
| Citric Acid | 0.14 | 0.16 | 0.16 | 0.16 | 0.17 | 0.18 |
| Anhydrous | | | | | | |
| Total | 100.0 | 100.0 | 100.00 | 100.00 | 100.00 | 100.00 |

*Removed during freeze drying.

Each of the pharmaceutical formulations provided in Table 8, above, were dosed as a 150 mg wet fill into a blister pocket designed to hold a 200 mg wet fill weight.

The batches were prepared by adding the gelatin, mannitol, and poloxamer (where applicable) to the purified water and heating to 60° C. while stirring with a magnetic follower. Once the gelatin had fully dissolved the solutions were cooled to 23° C. (±2° C.). at which point the glycopyrrolate was added followed by pH adjustment and addition of the cherry flavor and sucralose and the final alliquote of water to make the batch up to 100%.

A Hibar dosing pump was used to dose 150 mg in to 5 layer foil blister with preformed mold. The dosed pharmaceutical formulation was then frozen and then freeze dried.

The dosed trays were then frozen in a freeze tunnel set at −70° C. and a residence time of 3 minutes and 15 seconds and then transferred to a Refrigerated Freezer Cabinet (“RFC”) where it was held prior to freeze drying. A drying temperature of 0° C. was used and the product was dried for 6 hours.

Each sample was tested for pH, the results of which are provided below in Table 9.

TABLE 9

| | pH After |
|---|---|
| Batch | Adjustment |
| 1 | 4.60 |
| 2 | 4.48 |
| 3 | 4.50 |
| 4 | 4.53 |
| 5 | 4.49 |
| 6 | 4.47 |

The occurrence of minor wedging was also observed, the results of which are provided below in Table 10.

TABLE 10

| | HMW Fish | | |
|---|---|---|---|
| | Gelatin % | Poloxamer | |
| Batch | w/w | 188% w/w | % minor wedging |
| 1 | 5.00 | 0.10 | 33.7 |
| 2 | 5.00 | 0.025 | 68.8 |
| 3* | 5.25 | 0.05 | 51.3 |
| 4* | 5.25 | 0.05 | (same formulation - |
| | | | results combined) |
| 5 | 5.50 | 0.10 | 37.5 |
| 6 | 5.50 | 0.025 | 68.8 |

As shown in Table 10, there does not appear to be a correlation between gelatin level in the pharmaceutical composition and the occurrence of minor wedge shaped dosage forms prepared from pharmaceutical formulations comprising glycopyrrolate. However, as the amount of poloxamer 188 in the pharmaceutical formulation increases, the occurrence of wedge shaped dosage forms decreases.

### Testing Methods

Viscosity: A Haake VT550 viscotester was used to measure the pharmaceutical formulation solutions described above. The viscosity read at a shear rate of 500 sec⁻¹ with the temperature set to the same temperature as the temperature at which the mix was dosed.

Relative Density Testing: A Pycnometer was used to measure the relative density of pharmaceutical formulation solutions described above. The pycnometer determines density using the weight and the volume of the testing mix at 20° C. and comparing it to the weight and volume of purified water at 20° C. The relative density is determined using the following formula:

Relative density=(PMix−P′)/(PWater−P), where:
PMix=The weight of the pycnometer and test liquid, in mg.
P′=The weight of the empty pycnometer before weighting test liquid, in mg.
PWater=The weight of the pycnometer and water, in mg.
P=The weight of the empty pycnometer before weighting water, in mg.

Surface Tension Testing: A surface tension analyzer (DWK Life Sciences (Kimble) 14818 Tensiometer Capillary Surface Tension Apparatus) was used to determine the surface tension of pharmaceutical formulation solutions described above. The analyzer determines surface tension based on the height of liquid at 20° C. in a capillary tube according to the following formula:

Y=½(h)(r)(d)(g), where:
Y=surface tension (dynes/cm=mN/m)
h=distance between menisci (cm), average
r=radios of capillary (0.025 cm)
d=density of sample
g=acceleration due to gravity (980.7 cm/s2)

### Additional Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a formulation has at least about 10% w/w, about 15% w/w, or about 20% w/w is meant to mean that the formulation has at least about 10% w/w, at least about 15% w/w, or at least about 20% w/w.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A pharmaceutical formulation for preparing a pharmaceutical dosage form, the formulation comprising:
an active pharmaceutical ingredient;
0.01-0.3 wt. % of a surfactant;
4-6 wt. % of high molecular weight fish gelatin; and
a structure former.

2. The formulation of claim 1, wherein the surfactant comprises 0.05-0.2 wt. % of the pharmaceutical formulation

3. The formulation of claim 1, wherein the surfactant is a non-ionic surfactant.

4. The formulation of claim 3, wherein the non-ionic surfactant comprises polyoxyethylene-polyoxypropylene copolymer.

5. The formulation of claim 1, wherein the surfactant is poloxamer 188.

6. The formulation of claim 1, wherein the surfactant is an anionic surfactant.

7. The formulation of claim 6, wherein the anionic surfactant comprises one or more of sodium lauryl sulfate and docusate sodium.

8. The formulation of claim 1, wherein the pharmaceutical formulation comprises 4.5-5.5 wt. % of the high molecular weight fish gelatin.

9. The formulation of claim 1, wherein the pharmaceutical formulation comprises 3-5 wt. % of the structure former.

10. The formulation of claim 1, wherein the structure former comprises mannitol.

11. The formulation of claim 1, further comprising a pH modifier.

12. The formulation of claim 11, wherein the pH modifier comprises citric acid, maleic acid, tartaric acid, or hydrochloric acid.

13. The formulation of claim 1, wherein the pH of the pharmaceutical formulation is 4-6.

14. The formulation of claim 1, further comprising a solvent.

15. The formulation of claim 14, wherein the solvent comprises water.

16. The formulation of claim 1, wherein the active pharmaceutical ingredient comprises one or more of desmopressin and glycopyrrolate.

17. The formulation of claim 1, wherein the formulation has a viscosity of 9-12 mPa s.

18. The formulation of claim 1, wherein the formulation has a relative density of 1.2-1.3.

19. The formulation of claim 1, wherein the formulation has a surface tension of 60-80 mN/m.

20. A method of producing a freeze-dried dosage form for the delivery of an active pharmaceutical ingredient, the method comprises:
dosing a pharmaceutical formulation into a preformed mold, wherein the pharmaceutical formulation comprises:
an active pharmaceutical ingredient;
0.01-0.3 wt. % of a surfactant;
4-6 wt. % of high molecular weight fish gelatin; and
a structure former;
freeze-drying the dosed pharmaceutical formulation to form the dosage form.

21. The method of claim 20, further comprising freezing the dosed pharmaceutical formulation at a temperature of −40° C. to −120 ° C.

22. The method of claim 20, further comprising annealing the frozen pharmaceutical formulation by holding it at a temperature of less than −25° C. for 0.25-3 hours.

23. The method of claim 20, wherein the dosed pharmaceutical formulation is frozen at a temperature of −50° C. to −70° C. for a duration of about 1-5 minutes.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A dosage form for the delivery of an active pharmaceutical ingredient prepared by a process comprising:
dosing a pharmaceutical formulation into a preformed mold, wherein the pharmaceutical formulation comprises:
an active pharmaceutical ingredient;
0.01-0.3 wt. % of a surfactant;
4-6 wt. % of high molecular weight fish gelatin; and
a structure former;
freeze-drying the dosed pharmaceutical formulation to form the dosage form.

42. A dosage form comprising:
1.34-44.44 wt. % an active pharmaceutical ingredient;
0.13-1.33 wt. % of a surfactant;
26.67-53.62 wt. % of high molecular weight fish gelatin;
22.22-40.21 wt. % a structure former;
0.67-1.33 wt. % a pH modifier;
1.78-2.68 wt. % a sweetener; and
1.34-2.22 wt. % a flavoring agent.
